(19) United States
(12) Patent Application Publication
(10) Pub. No.: US 2025/0281443 A1
(43) Pub. Date: Sep. 11, 2025

(54) OIL-IN-WATER EMULSIONS AND USES THEREOF IN RESISTING INFANTILE ECZEMA

(71) Applicant: SHANDONG BENZHEN COSMETICS CO., LTD, Dezhou (CN)

(72) Inventors: Zhengmei HUANG, Dezhou (CN); Jinping DING, Dezhou (CN)

(73) Assignee: SHANDONG BENZHEN COSMETICS CO., LTD, Dezhou, SD (CN)

(21) Appl. No.: 18/556,825

(22) PCT Filed: Apr. 13, 2022
(86) PCT No.: PCT/CN2022/086724

§ 371 Date: Oct. 23, 2023

(30) Foreign Application Priority Data
Apr. 27, 2021 (CN) 202110458173.8

(51) Int. Cl. A61K 31/205; A61K 8/06; A61K 8/31; A61K 9/06; A61K 47/26; A61K 47/36; A61P 17/00

(52) U.S. Cl. CPC A61K 31/205; A61K 8/062; A61K 8/31; A61K 9/06; A61K 47/26; A61K 47/36; A61P 17/00

(57) ABSTRACT

It relates to an oil-in-water emulsion, comprising: Sclerotium Gum, petrolatum, water and optional water-soluble humectants selected from betaine, glycerin, sorbitol and/or a combination thereof. The water activity of the emulsion is 0.75 or less. It further relates to a kit comprising an oil-in-water emulsion, the kit containing a first moisturizing cream selected from the oil-in-water emulsion and a second aqueous solution containing water and optional water-soluble active ingredients. It relates to an application of the oil-in-water emulsion in resisting infantile eczema.

### FIELD OF TECHNOLOGY

The present disclosure relates to oil-in-water emulsions, particularly to emulsions for infantile eczema, which do not contain traditional preservatives and emulsifiers.

### BACKGROUND

Infants' skins are not as strong as mature skins, and more likely to increase epidermal water loss and stratum corneum hydration to result in reduced effectiveness of a skin barrier function. Because of a higher proportion between body surface and body weight of infants, topical medications are more easily absorbed and can have a more noticeable effect on the infants' skins. The skins of infants are more vulnerable to the environment than those of adults, become dry, and are prone to atopic dermatitis, diaper dermatitis and even infection.

There have been studies on inhibiting eczema statuses. For example, hormones are used, but hormones can bring a risk that the immunity of the local skin is reduced and meanwhile easily enter blood through transdermal absorption, thereby posing health risks. Infant creams contain synthetic ingredients, which pose a potential risk to infants.

Petrolatum such as vaseline are known to be an effective raw material to improve infantile eczema. However, the petrolatum is an oily substance with strong polarity, and can block pores to hinder the normal physiological function of the skin in addition to poor use experience if being directly used onto the skins of infants with eczema. When the petrolatum is used in a form of emulsion, it is generally needed to add emulsifiers and other ingredients, which can pose a potential health risk.

“STUDY ON NURSING EFFECT OF INFANT NECK ECZEMA” by Hu Yinyan discloses a research on combined treatment and nursing of infantile eczema with vaseline and traditional Chinese medicines. The combined treatment of vaseline strips and traditional Chinese medicine needs high medical knowledge to be guided.

CN106075120A discloses an eczema cream comprising: 25-35 parts of β-glucan, 2-10 parts of purslane extract, 0.1-2 parts of notoginseng triterpenes, 5-20 parts of natural vegetable oil, 2-6 parts of emulsifier and 20-80 parts of purified water. The eczema cream realizes the effect of removing eczema through β-glucan, purslane extract, panax notoginsenosides and natural vegetable oil. However, many emulsifiers are used in this product, which poses a potential threat to infant skins.

CN1466952A discloses that laminarin is a hydrocarbon which belongs to β-glucan. The laminarin exists in mushrooms such as shiitake mushrooms, seaweeds such as kelp, and has the effect of improving immunity. It is known that it is difficult to be separately absorbed, and suitably ingested together with proteins. However, this product does not disclose how to be applied to treatment of infantile eczema.

CN106176507A discloses an eczema-removal emulsion containing: 0.1-3 parts of Centella asiatica extract, 0.1-1 part of Marrubium vulgare extract, 0.1-5 parts of yeast β-glucan and 0.1-3 parts of dragon head bamboo extract. This product uses medicinal plant extracts and other ingredients as active ingredients to alleviate eczema. For infant products, their safety are not guaranteed.

CN109431943A discloses a repairing cream for infantile eczema, comprising: holy basil extract, Avena strigosa seed extract, inverted bell extract, Ribes nigrum extract, blue thistle oil, moringa oleifera seed oil, Echinacea purpurea extract, egg yolk oil, emollient, moisturizer, antioxidant, antiallergic agent, thickener, emulsifier, co-emulsifier, preservative, etc. This product contains the preservative, flavors, the emulsifier and the like which possess potential safety risks for infant skins.

CN108771649A discloses a baby redoubling-moisturizing cream containing shea butter, comprising: shea butter fruit fat, betaine, trehalose, flavor, rhodiola, Centella asiatica extract, Polygonum cuspidatum root extract, scutellaria root extract, wild chrysanthemum extract, etc. The moisturizing cream containing shea butter and other ingredients can redouble to moisturize the skins of the children to realize deep nourishing and moisturizing. But this product contains preservatives, flavors, emulsifiers and the like which possess other potential safety risks for infant skins.

CN108778240A discloses an infant skin care product, which comprises medical grade water, at least one stabilizer, at least one nutrient and at least one emulsifier. The infant skin care product can also comprise zinc gluconate, magnesium aspartate, copper gluconate (composite trace element) and Dunaliella salina extract, among them, stable ingredients include shea butter and other ingredients, but the product contains emulsifiers, which have potential safety risks to infant skin.

Therefore, products that do not contain emulsifiers or preservatives have been developed to deal with the potential threat brought by emulsifiers, flavors and preservatives.

CN109431838A discloses that an essence product that does not contain emulsifiers is prepared by compounding behenyl alcohol, cetearyl alcohol and dipalmitoyl hydroxyproline to replace the traditional emulsifiers. However, this product is mainly an aqueous solution, in which only a small amount of oil phase components and substances with preservative functions such as 1, 2-pentanediol are used, and these ingredients have no good inhibition effect on eczema.

CN111264863A discloses that an emulsion is prepared by using a composite formed by proanthocyanidins and oat β-glucan as an emulsifier, the prepared emulsion has attractive reddish-brown appearance and stability, and meanwhile has the function of protecting functional oil or fat soluble biological active ingredients. However, this product is not publicly used for removing infantile eczema.

CN111346053A discloses that an emulsifier is prepared by using β-glucan, which is a stable emulsion containing olive oil. β-glucan is cultured and obtained by using a special preparation method, but it cannot be determined whether the emulsifier is suitably used as an oil ingredient for infantile eczema and whether additional stable ingredients such as preservatives are required.

US2011168055A1 discloses an emulsion that contains (1→3)-β-D-glucan based on the total weight of 0.01 to 10 weight parts of the emulsion. The emulsion contains a bituminous binder which is not disclosed to be used for resisting infantile eczema.

Researches on emulsifying property of oat β-glucan by Shen Ruiling et al. disclose that the glucan has a certain emulsifying ability and can be used for preparing emulsifiers. However, it is uncertain whether it has the effect of resisting eczema by not disclosing how to prepare products without preservatives.

US2012045422A1 discloses a method for treating or preventing eczema in a subject, which includes administering Lactobacillus rhamnosus to a subject in need.

WO2020191620A1 has disclosed that an exemplary natural skin care emulsion without preservatives contains 5-30 parts of glycerol, 5-30 parts of betaine, 5-20 parts of xylitol, 5-30 parts of fructose, 30-40 parts of plant extracts and 0.1-0.5 parts of xanthan gum. The oil phase contains 1-2 parts of soybean lecithin, 5-15 parts of shea butter, 5-15 parts of olive oil and 1-10 parts of sea buckthorn oil. This product still contains an emulsifier ingredient, and it cannot be determined whether it is effective for infantile eczema.

Emulsifiers used in lotions and creams of infant products can emulsify the sebum in the infant stratum corneum, causing the water to wash away the stratum corneum sebum and destroying the protective barrier function of the skin. Exogenous substances can cause skin irritation and lead to anaphylaxis. All types of perfumes and dyes used currently have the risk of sensitization and allergic reactions.

Therefore, it is still necessary to find a safe and effective product suitable for infants in treating eczema.

### SUMMARY

One objective of the present disclosure is to provide a low irritation, more safe and stable and long term storable skin care product, which has a good improvement effect on infantile eczema.

The inventors unexpectedly found that the stability of an emulsion with high content of the petrolatum or shea butter can be stabilized by Sclerotium Gum, so as to prepare the petrolatum or shea butter into the emulsion, thereby avoiding the use of emulsifier components and improving use experience and eczema-resisting effects.

Further, the inventors unexpectedly found that the emulsions prepared from Sclerotium Gum and water as well as water-soluble components selected from betaine, glycerol, sorbitol and/or a combination thereof, having a water activity of 0.75 or less, are effective in controlling the existence of microorganisms, thereby avoiding the use of preservative ingredients.

Therefore, in one aspect, the present disclosure provides an oil-in-water emulsion, based on the total weight of the emulsion, comprising:
(a) 45-65 parts by weight, preferably 50-60 parts by weight, especially preferably 55 parts by weight, of betaine;
(b) 0.1-2 parts by weight, preferably 0.1-1 parts by weight, especially preferably 0.18 parts by weight, of Sclerotium Gum;
(c) 35-55 parts by weight, preferably 40-50 parts by weight, especially preferably 45 parts by weight, of water; and
(d) 5-90 parts by weight, preferably 10-40 parts by weight, especially preferably 15 parts by weight, of petrolatum;
the emulsion having a water activity of 0.75 or less.

Further, the present disclosure relates to an oil-in-water emulsion, based on the total weight of the emulsion, comprising:
(a) 25-60 parts by weight, preferably 40-50 parts by weight, especially preferably 50 parts by weight, of betaine;
(b) 0.1-30 parts by weight, preferably 10-20 parts by weight, especially preferably 20 parts by weight, of glycerin;
(c) 0.1-2 parts by weight, preferably 0.1-1 parts by weight, especially preferably 0.18 part by weight, of Sclerotium Gum;
(d) 5-60 parts by weight, preferably 10-40 parts by weight, especially preferably 15 parts by weight, of petrolatum; and
(e) 30-50 parts by weight, preferably 30-40 parts by weight, especially preferably 30 parts by weight, of water;
the emulsion having a water activity of 0.75 or less.

Further, the present disclosure relates to an oil-in-water emulsion, based on the total weight of the emulsion, comprising
(a) 65-85 parts by weight, preferably 70-85 parts by weight, especially preferably 70 parts by weight, of sorbitol;
(b) 0.1-2 parts by weight, preferably 0.1-1 parts by weight, especially preferably 0.18 part by weight, of Sclerotium Gum;
(c) 5-90 parts by weight, preferably 10-40 parts by weight, especially preferably 15 parts by weight, of petrolatum; and
(d) 15-35 parts by weight, preferably 10-30 parts by weight, especially preferably 30 parts by weight, of water; and
the emulsion having a water activity of 0.75 or less.

Further, the present disclosure relates to an oil-in-water emulsion, based on the total weight of the emulsion, comprising:
(a) 40-85 parts by weight, preferably 40-80 parts by weight, especially preferably 50 parts by weight, of sorbitol;
(b) 0.1-30 parts by weight, preferably 10-30 parts by weight, especially preferably 20 parts by weight, of glycerin;
(c) 0.1-2 parts by weight, preferably 0.1-1 parts by weight, especially preferably 0.18 part by weight, of Sclerotium Gum;
(d) 5-60 parts by weight, preferably 10-40 parts by weight, especially preferably 15 parts by weight, of petrolatum; and
(e) 15-40 parts by weight, preferably 20-35 parts by weight, especially preferably 30 parts by weight, of water;
the emulsion having a water activity of 0.75 or less.

Further, the present disclosure relates to an oil-in-water emulsion, based on the total weight of the emulsion, comprising:
(a) 0.1-75 parts by weight, preferably 10-70 parts by weight, especially preferably 50 parts by weight, of sorbitol;
(b) 0.1-65 parts by weight, preferably 10-60 parts by weight, especially preferably 20 parts by weight, of betaine;
(c) 0.1-2 parts by weight, preferably 0.1-1 parts by weight, especially preferably 0.18 part by weight, of Sclerotium Gum;
(d) 5-70 parts by weight, preferably 10-40 parts by weight, especially preferably 15 parts by weight, of petrolatum; and
(e) 25-55 parts by weight, preferably 30-50 parts by weight, especially preferably 30 parts by weight, of water;
the emulsion having a water activity of 0.75 or less.

Further, for any one of the above oil-in-water emulsions, they do not contain a preservative.

Further, for any one of the above oil-in-water emulsions, they do not contain a surfactant.

Further, the present disclosure also relates to a kit, comprising:
a first moisturizing cream selected from any one of the oil-in-water emulsion of the present disclosure; and
a second aqueous solution containing 95-100 parts by weight of water and 0-5 parts by weight of optional water-soluble active ingredients.

Further, the present disclosure also relates to a method for caring for skins, comprising the following steps:
(1) taking the first moisturizing cream;
(2) adding the second aqueous solution which is 2-10 times by weight of the first moisturizing cream, and uniformly mixing to obtain a mixture; and
(3) evenly smearing the mixture obtained in step (2) on skins;
the steps (1) and (2) can be implemented on skins in turn or simultaneously.

Further, the present disclosure relates to an application of the oil-in-water emulsion in preparation of cosmetics and/or medicines for infantile eczema.

### Sclerotium Gum

Sclerotium rolfsii is a fungus existing in soil. It produces Sclerotium Gum via fermentation. The Sclerotium Gum is a common cosmetic ingredient and can be widely applied. The Sclerotium Gum is most commonly used as a thickener or a gelling agent, has high thermal stability, can help to enhance the moisture barrier of the skin, prevents moisture loss through epidermis and helps to keep the skin moist.

The Sclerotium Gum used in the present disclosure has the following chemical structure:

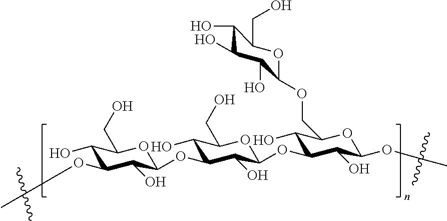

According to the requirement for stabilizing the oil-in-water emulsion, the amount of the Sclerotium Gum is based on parts by weight, the oil-in-water emulsion contains 0.1-2 parts by weight, preferably 0.1-1 weight, especially preferably 0.18 part by weight, of Sclerotium Gum.

The Sclerotium Gum suitable in the present application, has a viscosity of greater than 1000 mPa·s when its active matter content is 0.5%, preferably greater than 2000 mPa·s, especially preferably greater than 4000 mPa·s, especially the Sclerotium Gum produced by Onlystar.

### Petrolatum

The petrolatum (also referred to as vaseline) has a compositions being between C₁₇H₃₆ and C₂₁H₄₄ and is a high molecular hydrocarbon obtained from petroleum fractions with a distillation end temperature above 303° C. The petrolatum has a specific gravity of between 0.820 and 0.865 and is insoluble in water, but is easily soluble in gasoline and pine oil.

The vaseline prepared by deep refining is a colorless, odorless, non-fluorescent and transparent oily liquid with a melting point of about 37-54° C., does not contain any additives, moisture and mechanical impurities, and is insoluble in water.

The vaseline is an alkane, and therefore has extremely stable chemical property, good anti-oxidation safety, stability and light safety. The vaseline cannot be utilized by microorganisms, is difficult to decay and deteriorate, and is not easily generate character change.

Based on requirements for closing skins and repairing eczema, the petrolatum of the present disclosure preferably uses pharmaceutical grade vaseline. In parts by weight, the oil-in-water emulsion contains 5.0-25.0 parts by weight, preferably 10-25 parts by weight, especially preferably 15-20 parts by weight, of petrolatum.

### Water-Soluble Active Ingredient

The present disclosure further optionally contains water-soluble active ingredients without special limitations. The water-soluble active ingredients of the present disclosure comprise water-soluble active ingredients beneficial to improving skin conditions, for example prebiotics derived from a lactic acid extract, and its amount is 0-5 parts by weight.

According to an embodiment of the present disclosure, the water-soluble active ingredient can exist in the first moisturizing cream or the second aqueous solution alone or simultaneously.

### Surfactant

Further, the oil-in-water emulsion of the present disclosure does preferably not contain surfactants. The term “surfactant” of the present disclosure is also referred to as an interfacial surfactant, which is generally a substance having an emulsifying effect in the present disclosure, and is a substance capable of reducing the surface tension of the liquid or the interfacial tension between two phases. The surfactant is amphiphilic and contains hydrophilic and lipophilic groups. Examples of hydrophilic groups include but are not limited to amino groups, carboxyl groups, sulfonic groups, hydroxyl groups, etc., and examples of hydrophobic groups include but are not limited to aliphatic alkanes, olefins, alkynes and aromatic hydrocarbons of C8 and above. Examples of commonly used surfactants include, but are not limited to, natural surfactants or derivatives thereof and synthetic surfactants. Examples of natural surfactants or derivatives thereof are protein based surfactants.

Exemplary anionic surfactants, for example, water-soluble salts of higher fatty acid monoglyceride monosulfate, such as sodium salts of monosulfated monoglyceride of hydrogenated coconut oil fatty acid, such as sodium N-methyl N-coconut acyl taurine and sodium coconut oil glyceride sulfate; higher alkyl sulfate, such as sodium lauryl sulfate; higher alkyl ether sulfates, such as sodium lauryl alcohol polyether-2 sulfate; higher alkyl aryl sulfonates, such as sodium dodecylbenzene sulfonate (sodium lauryl benzene sulfonate); Higher alkyl sulfoacetates, such as sodium lauryl sulfoacetate (sodium dodecyl sulfoacetate), higher fatty acid esters of 1,2-dihydroxypropane sulfonic acid, sulfonyl laurate and sodium lauryl sarcosinate.

The cationic surfactants described in the present disclosure can be broadly defined as derivatives of aliphatic quaternary ammonium compounds with a long alkyl chain containing 8-18 carbon atoms, such as lauryl trimethylammonium chloride, cetyl pyridine chloride, cetyl trimethylammonium bromide, diisobutyl phenoxyethyl dimethyl benzyl ammonium chloride, coconut alkyl trimethylammonium nitrite, cetyl pyridine fluoride and their mixtures.

Exemplary non-ionic surfactants can be broadly defined as compounds produced by condensation of alkene oxide groups with organic hydrophobic compounds that can be essentially aliphatic or alkyl aromatic compounds. Examples of suitable nonionic surfactants include, but are not limited to, polyethylene oxide condensates of alkyl phenols, condensation products derived from the reaction products of ethylene oxide with propylene oxide and ethylenediamine, ethylene oxide condensates of aliphatic alcohols, long-chain tertiary amine oxides, long-chain tertiary phosphine oxides, long-chain dialkyl sulfoxides, and mixtures of such substances, such as poloxamer, polysorbate and polyethylene glycol hydrogenated castor oil.

Exemplary amphoteric surfactants include: betaine (such as coconut amide propyl betaine); derivatives of aliphatic secondary and tertiary amines, wherein the aliphatic group can be straight or branched chain, and one of the aliphatic substituents contains about 8-18 carbon atoms and one of the aliphatic substituents contains anionic water solubilizing groups (such as carboxylate, sulfonate, sulfate, phosphate or phosphonate); and mixtures of such the materials.

Further, the emulsion of the present disclosure does not contain preservatives and bacteriostatic agents. The preservatives and bacteriostatic agents are selected from alcohols, formaldehyde donors and aldehyde derivatives, benzoic acid and its derivatives and other organic compounds. For example, phenoxyethanol, benzyl alcohol, imidazolidinyl urea, p-hydroxybenzoic acid esters, bronopol, carson and quaternary ammonium salts, etc.

Without theoretical restriction, the water activity of the emulsion is adjusted to no more than 0.75 through water, betaine and optional water-soluble polyols and/or water-soluble polysaccharides to inhibit the growth of microorganisms. Therefore, the water activity of the oil-in-water emulsion of the present disclosure is less than or equal to 0.75, preferably less than or equal to 0.65, especially preferably less than or equal to 0.6.

The oil-in-water emulsion according to the present disclosure can thicken the skin barrier layer of the infant, improve the moisture retention ability of the skin, and prevent the external risk ingredients from entering the skin to cause allergy. The emulsion material of the present disclosure is inert and safe, and can prevent formula ingredients from permeating into the skin to cause allergy.

The present disclosure does not use emulsifiers, so as to reduce the damage to the skin barrier caused by emulsifiers. The present disclosure does not use bacteriostatic agents or preservatives to protect the microbiota on the infant skin.

### BRIEF DESCRIPTION OF THE DRAWINGS

FIG. 1 is an infrared spectrum of Sclerotium Gum used in the present disclosure.

FIG. 2 shows emulsion stability of different Sclerotium Gum systems.

FIG. 3 shows emulsion stability of different Sclerotium Gum systems.

FIG. 4 is a microscope image of example 1 (1-3 and blank control).

FIG. 5 shows use of a moisturizing cream in example 6 for back eczema of three-month-old infants.

FIG. 6 shows use of a moisturizing cream in example 6 for facial eczema of 42-day-old infants.

FIG. 7 shows use of a moisturizing cream in example 6 for facial eczema of 4-month-old infants.

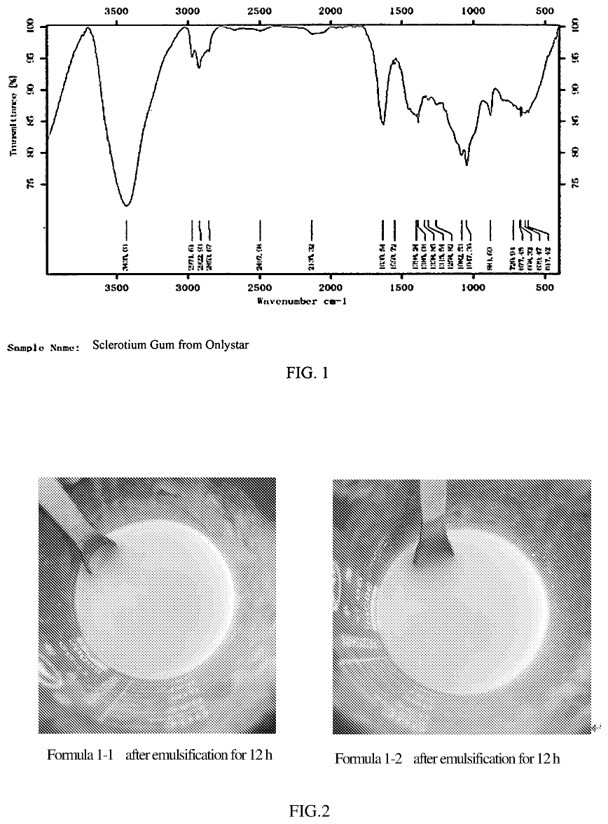

### DESCRIPTION OF THE EMBODIMENTS

The present disclosure is further described below in combination with specific embodiments. It should be understood that these embodiments are only used to illustrate the disclosure and not to limit the scope of the present disclosure. The experimental method without specific conditions in the following embodiments is usually in accordance with the conventional conditions or the conditions recommended by the manufacturer. Unless otherwise stated, percentages and portions are weight percentages and weight portions.

The experimental methods used in the following embodiments are conventional methods unless otherwise specified.

The materials, reagents, equipment and the like used in the following embodiments can be obtained from commercial sources without special instructions.

### Example 1: Oil-In-Water Emulsions and the Stability Test

The oil-in-water emulsions are prepared respectively through a formula in Table 1 (in parts by weight).

TABLE 1

| Experiment number | 1-1 | 1-2 | 1-3 | Blank control |
|---|---|---|---|---|
| Water | 38 | 50 | 45 | 45 |
| Betaine | 62 | 50 | 55 | 55 |
| Sclerotium Gum | 0.18 | 0.18 | 0.18 | 0 |
| Petrolatum | 15 | 15 | 25 | 15 |
| Stability conclusion | + | + | + | − |

### Stability Test

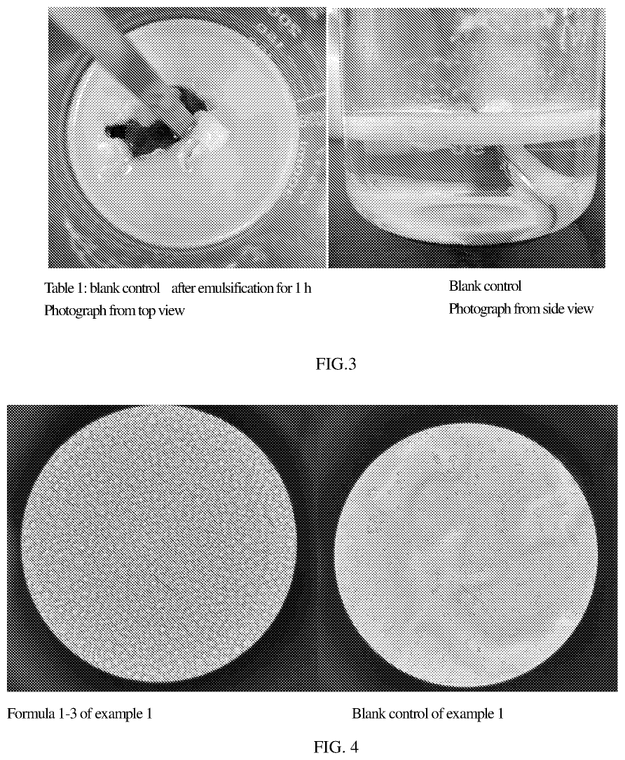

The prepared samples were put at room temperature for 24 h to observe the stability, that is, to observe whether oil droplets were separated out. If no oil droplets were separated out, it is determined that the stability is passed (+), or else the stability is not passed (−). The results are as shown in FIGS. 2-3.

Centrifugation experiment: the prepared samples were pre-heated in a 38° C. constant-temperature incubator for 1 h, and then transferred to a centrifuge to be centrifuged for 30 min at 2000 r/min, and the samples centrifuged were taken out for observation. If there is no delamination, it is determined that the stability is passed, or else the stability is not passed.

### Heat Resistance and Cold Resistance Experiments

### Heat Resistance Experiment:

The prepared samples were put in a 50° C. constant-temperature incubator, and taken out after 6 weeks to observe whether there is oil-water delamination. If there is no oil-water delamination, it is determined that the stability is passed (+), or else the stability is not passed (−).

Cold resistance experiment: the prepared samples were put at −18° C. for 24 h, then taken out and placed at room temperature for 24 h, and the above steps were repeated 6 times. If no demulsification is found, it is determined that the stability is passed (+), or else the stability is not passed (−).

Microscope examinations for formula 1-3 and blank control show that the oil-in-water emulsion with Sclerotium Gum can form homogeneous oil droplets, while the oil droplet of the oil-in-water emulsion without Sclerotium Gum has uneven particle size distribution of oil droplet.

Stability study was performed on the following examples 2-5 according to the above stability text method.

### Example 2: Oil-In-Water Emulsions and the Stability Test

The oil-in-water emulsions are prepared through formulas in Table 2 (in parts by weight).

TABLE 2

| Experiment number | 2-1 | 2-2 | 2-3 | 2-4 | 2-5 | Blank control |
|---|---|---|---|---|---|---|
| Water | 30 | 30 | 50 | 50 | 30 | 30 |
| Betaine | 60 | 50 | 50 | 25 | 60 | 50 |
| Glycerin | 10 | 20 | | 25 | 10 | 20 |
| Sclerotium Gum | 0.18 | 0.18 | 0.18 | 0.18 | 0.18 | 0 |
| Petrolatum | 15 | 15 | 15 | 15 | 50 | 15 |
| Stability conclusion | + | + | + | + | + | − |

### Example 3: Oil-In-Water Emulsions and the Stability Test

The oil-in-water emulsions are prepared through formulas in Table 3 (in parts by weight).

TABLE 3

| Experiment number | 3-1 | 3-2 | 3-3 | 3-4 | Stability conclusion |
|---|---|---|---|---|---|
| Water | 15 | 30 | 20 | 15 | 30 |
| Sorbitol | 85 | 70 | 80 | 85 | 70 |
| Sclerotium Gum | 0.18 | 0.18 | 0.2 | 0.25 | 0 |
| Petrolatum | 15 | 15 | 70 | 90 | 15 |
| Stability conclusion | + | + | + | + | − |

### Example 4: An Oil-In-Water Emulsions and the Stability Test

The oil-in-water emulsions are prepared through formulas in Table 4 (in parts by weight).

TABLE 4

| Experiment number | 4-1 | 4-2 | 4-3 | 4-4 | 4-5 | Blank control |
|---|---|---|---|---|---|---|
| Water | 30 | 20 | 30 | 30 | 20 | 30 |
| Glycerin | 20 | | 10 | 30 | | 20 |
| Sorbitol | 50 | 80 | 60 | 40 | 80 | 50 |
| Sclerotium Gum | 0.18 | 0.18 | 0.18 | 0.18 | 0.18 | 0 |
| Petrolatum | 15 | 15 | 15 | 15 | 40 | 15 |
| Stability conclusion | + | + | + | + | + | − |

### Example 5: Oil-In-Water Emulsions and the Stability Test

The oil-in-water emulsions are prepared through formulas in Table 5 (in parts by weight).

TABLE 5

| Experiment number | 5-1 | 5-2 | 5-3 | 5-4 | 5-5 | Blank control |
|---|---|---|---|---|---|---|
| Water | 30 | 30 | 30 | 50 | 50 | 30 |
| Sorbitol | 60 | 10 | 50 | 49 | 1 | 50 |
| Betaine | 10 | 60 | 20 | 1 | 49 | 20 |
| Sclerotium Gum | 0.18 | 0.18 | 0.18 | 0.18 | 0.18 | 0 |
| Petrolatum | 15 | 15 | 15 | 15 | 15 | 15 |
| Stability conclusion | + | + | + | + | + | − |

### Example 6: A Preparation Method and Application Method of a Kit for Infantile Eczema

Formula of the first moisturizing cream: 30 parts by weight of water, 50 parts by weight of sorbitol, 20 parts by weight of betaine, 0.18 part by weight of Sclerotium Gum, 15 parts by weight of petrolatum and 0.5 part by weight of lactic acid extract.

The second aqueous solution: 100 parts by weight of purified water.

According to the application method of the present disclosure: 0.5 g of moisturizing cream was taken, 1 g of water was added onto a palm, the moisturizing cream and the water were uniformly mixed and then applied to the inner side of an arm to evaluate skin feeling.

Control experiment: the first moisturizing cream was directly applied onto the inner side of the arm to evaluate the skin feeling.

The skin feeling comfort is taken as a standard, dryness and moisturizing represents comfortable, sticky and airtight represent uncomfortable. Comfortable: 5 scores, relatively comfortable: 4 scores, average: 3 scores, uncomfortable: 2 scores, extreme uncomfortable: 1 score.

Results are as shown in the Table below.

Evaluation of skin feeling

| Evaluation experts | The present disclosure | Control |
|---|---|---|
| 1 | 5 | 1 |
| 2 | 5 | 1 |
| 3 | 4 | 2 |
| 4 | 5 | 1 |
| 5 | 4 | 3 |
| Average scores | 4.6 | 1.6 |

### Effect on Infantile Eczema

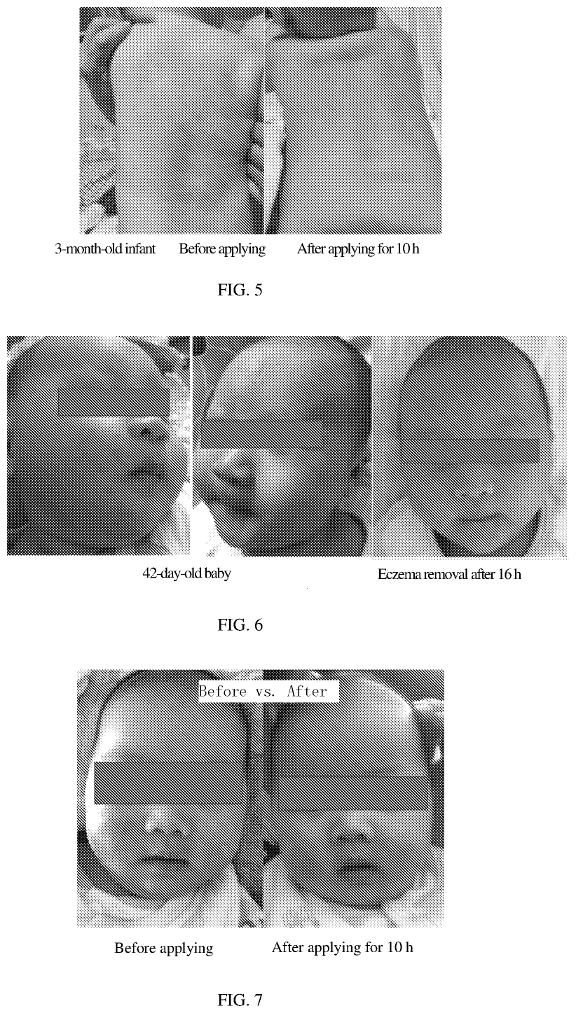

According to the application method of the present disclosure, the moisturizing cream is applied to the affected part of infantile eczema to evaluate the effect of removing infantile eczema. As shown in FIGS. 5-7, the moisturizing cream of the present disclosure has a good inhibitory effect on infantile eczema.

### Anti-Corrosion Challenging Experiment

This experiment refers to the microbiological antiseptic efficacy test method of USP39<51> in the United States Pharmacopoeia, that is, 30 g of each test sample was weighed into a sterilized sample bottle, a quantitative mixed bacterial suspension was added, and the initial bacterial concentration in the sample was 10⁶ cfu/g; the initial concentrations of yeast and mold are 10⁵ cfu/g. The above substances were uniformly mixed, the mouth of the sample bottle was sealed with sealing glue and then the sample bottle was placed in an incubator at about 28° C. Samples were taken and analyzed at 7, 14, 21 and 28 days after inoculation.

### Result Analysis

According to the following table, the result of the microbial challenge tests of samples of the two emulsion anticorrosive systems in examples 1-1# and 1-2# show that the emulsion can pass the challenge test in personal care products. Accordingly, the personal care emulsion of the present disclosure can take a good anti-corrosive effect on the formula.

| | Compounded | | | | | | |
|---|---|---|---|---|---|---|---|
| | formula | Total bacterial colonies/(cfu · g⁻¹) | | | | | |
| Strains | number | Day 0 | Day 1 | Day 7 | Day 14 | Day 21 | Day 28 |
| Mixed | Example 1-1# | 1.2 × 10⁶ | 3.7 × 10⁴ | 5.0 × 10³ | 2.0 × 10² | 1.5 × 10² | 1.0 × 10² |
| strain | Example 1-2# | 1.4 × 10⁶ | 4.0 × 10⁴ | 4.7 × 10³ | 3.0 × 10² | 1.0 × 10² | 80 |
| Mixed | Example 1-1# | 1.6 × 10⁶ | 3.3 × 10⁴ | 3.0 × 10³ | 1.0 × 10³ | 3.0 × 10² | 2.0 × 10² |
| fungi | Example 1-2# | 2.1 × 10⁶ | 3.0 × 10⁴ | 3.6 × 10³ | 1.2 × 10³ | 2.1 × 10² | 1.1 × 10² |

1. An oil-in-water emulsion, comprising, based on the total weight of the emulsion,
(a) 45-65 parts by weight, preferably 50-60 parts by weight, especially preferably 55 parts by weight, of betaine;
(b) 0.1-2 parts by weight, preferably 0.1-1 parts by weight, especially preferably 0.18 part by weight, of Sclerotium Gum;
(c) 35-55 parts by weight, preferably 40-50 parts by weight, especially preferably 45 parts by weight, of water; and
(d) 5-90 parts by weight, preferably 10-40 parts by weight, especially preferably 15 parts by weight, of petrolatum;
the emulsion having a water activity of 0.75 or less.

2. An oil-in-water emulsion, comprising, based on the total weight of the emulsion,
(a) 25-60 parts by weight, preferably 40-50 parts by weight, especially preferably 50 parts by weight, of betaine;
(b) 0.1-30 parts by weight, preferably 10-20 parts by weight, especially preferably 20 parts by weight, of glycerin;
(c) 0.1-2 parts by weight, preferably 0.1-1 parts by weight, especially preferably 0.18 part by weight, of Sclerotium Gum;
(d) 5-60 parts by weight, preferably 10-40 parts by weight, especially preferably 15 parts by weight, of petrolatum; and
(e) 30-50 parts by weight, preferably 30-40 parts by weight, especially preferably 30 parts by weight, of water;
the emulsion having a water activity of 0.75 or less.

3. An oil-in-water emulsion, comprising, based on the total weight of the emulsion,
(a) 65-85 parts by weight, preferably 70-85 parts by weight, especially preferably 70 parts by weight, of sorbitol;
(b) 0.1-2 parts by weight, preferably 0.1-1 parts by weight, especially preferably 0.18 part by weight, of Sclerotium Gum;
(c) 5-90 parts by weight, preferably 10-40 parts by weight, especially preferably 15 parts by weight, of petrolatum; and
(d) 15-35 parts by weight, preferably 10-30 parts by weight, especially preferably 30 parts by weight, of water;
the emulsion having a water activity of 0.75 or less.

4. An oil-in-water emulsion, based on the total weight of the emulsion, comprising, based on the total weight of the emulsion,
(a) 40-85 parts by weight, preferably 40-80 parts by weight, especially preferably 50 parts by weight, of sorbitol;
(b) 0.1-30 parts by weight, preferably 10-30 parts by weight, especially preferably 20 parts by weight, of glycerin;
(c) 0.1-2 parts by weight, preferably 0.1-1 parts by weight, especially preferably 0.18 part by weight, of Sclerotium Gum;
(d) 5-60 parts by weight, preferably 10-40 parts by weight, especially preferably 15 parts by weight, of petrolatum; and
(e) 15-40 parts by weight, preferably 20-35 parts by weight, especially preferably 30 parts by weight, of water;
the emulsion having a water activity of 0.75 or less.

5. An oil-in-water emulsion, comprising, based on the total weight of the emulsion,
(a) 0.1-75 parts by weight, preferably 10-70 parts by weight, especially preferably 50 parts by weight, of sorbitol;
(b) 0.1-65 parts by weight, preferably 10-60 parts by weight, especially preferably 20 parts by weight, of betaine;
(c) 0.1-2 parts by weight, preferably 0.1-1 parts by weight, especially preferably 0.18 part by weight, of Sclerotium Gum;
(d) 5-70 parts by weight, preferably 10-40 parts by weight, especially preferably 15 parts by weight, of petrolatum; and
(e) 25-55 parts by weight, preferably 30-50 parts by weight, especially preferably 30 parts by weight, of water;
the emulsion having a water activity of 0.75 or less.

6. The oil-in-water emulsion according to claim 1, which does not contain a preservative.

7. The oil-in-water emulsion according to claim 1, which does not contain a surfactant.

8. A kit, comprising:
a first moisturizing cream selected from the oil-in-water emulsion according to claim 1; and
a second aqueous solution containing 95-100 parts by weight of water and 0-5 parts by weight of optional water-soluble active ingredients;
the kit being applied by the following steps:
(1) taking the first moisturizing cream;
(2) adding the second aqueous solution which is 2-10 times by weight of the first moisturizing cream, and uniformly mixing to obtain a mixture; and
(3) evenly smearing the mixture obtained in step (2) on skins;
the steps (1) and (2) being implemented in turn or simultaneously.

9. An application of the oil-in-water emulsion according to claim 1 in preparation of cosmetics and/or medicines for infantile eczema.

10. The oil-in-water emulsion according to claim 2, which does not contain a preservative.

11. The oil-in-water emulsion according to claim 3, which does not contain a preservative.

12. The oil-in-water emulsion according to claim 4, which does not contain a preservative.

13. The oil-in-water emulsion according to claim 5, which does not contain a preservative.

14. The oil-in-water emulsion according to claim 2, which does not contain a surfactant.

15. The oil-in-water emulsion according to claim 3, which does not contain a surfactant.

16. The oil-in-water emulsion according to claim 4, which does not contain a surfactant.

17. The oil-in-water emulsion according to claim 5, which does not contain a surfactant.

18. The oil-in-water emulsion according to claim 6, which does not contain a surfactant.
